(19) United States
(12) Patent Application Publication
(10) Pub. No.: US 2009/0192228 A1
(43) Pub. Date: Jul. 30, 2009

(54) Controlled-Release Tolterodine Compositions and Methods

(75) Inventor: Hui Wang, Warren, NJ (US)

Correspondence Address: CANTOR COLBURN, LLP, 20 Church Street, 22nd Floor, Hartford, CT, 06103, US

(73) Assignee: ACTAVIS GROUP PTC EHF, Hafnarfjordur (IS)

(21) Appl. No.: 12/360,254

(22) Filed: Jan. 27, 2009

Related U.S. Application Data
(60) Provisional application No. 61/023,907, filed on Jan. 28, 2008.

(51) Int. Cl. A61K 31/135

(52) U.S. Cl. 514/648

(57) ABSTRACT

Controlled-release tolterodine compositions, including controlled-release particles, pellets, granules, and spheres, comprising an inert core, a first layer disposed on the inert core, and a second layer disposed on the first layer are described. Methods for preparing such compositions and methods of treating a variety of disorders are also disclosed.

### CROSS REFERENCE TO RELATED APPLICATIONS

This application claims priority from Provisional Application Ser. No. 61/023,907, filed Jan. 28, 2008, which is incorporated herein by reference in its entirety.

### BACKGROUND

Tolterodine, (R)—N,N-diisopropyl-3-(2-hydroxy-5-methylphenyl)-3-phenylpropanamine, has been marketed for the treatment of urge incontinence and other symptoms of unstable or overactive urinary bladder. Tolterodine is equipotent in the bladder with oxybutynin, another drug used to treat incontinence, however, its affinity for muscarinic receptors of the salivary gland is eight times lower than that of oxybutynin. Both tolterodine and its major active metabolite, the 5-hydroxymethyl derivative of tolterodine, have considerably fewer side-effects than oxybutynin, particularly a reduced propensity to cause dry mouth.

Both immediate release and controlled-release compositions are currently available for the administration of tolterodine. The immediate release composition is a film coated tablet containing 1 mg or 2 mg of tolterodine L-tartrate administered at a strength of 2 mg twice a day. Controlled-release compositions of tolterodine have been produced to reduce dosing frequencies and improve side effect profiles. For example, U.S. Pat. No. 6,911,217 describes controlled-release beads comprising a core unit of a substantially water soluble or water swellable inert material, a first layer (“sealcoat”) on the core unit of a substantially water-insoluble polymer, a second layer covering the first layer and containing tolterodine, and a third layer of polymer on the second layer effective for controlled-release of the tolterodine.

There is need for simpler controlled-release tolterodine compositions that can provide a controlled-release profile and/or reduced side effects.

### SUMMARY

The above-described and other deficiencies are met by a controlled-release composition comprising (i) an inert core comprising a water insoluble polymer; (ii) a first layer disposed on the inert core, wherein the first layer comprises tolterodine or a pharmaceutically acceptable salt thereof and a binder; and (iii) a second layer disposed on the first layer, wherein the second layer comprises a water insoluble polymer, a plasticizer, and a pore-forming agent.

In another embodiment, a controlled-release composition comprises (i) an inert core consisting essentially of a water insoluble polymer; (ii) a first layer coated on the inert core, wherein the first layer consists essentially of tolterodine or a pharmaceutically acceptable salt thereof and a binder; and (iii) a second layer coated on the first layer, wherein the second layer consists essentially of a water insoluble polymer, a plasticizer, and a pore-forming agent.

Also disclosed are methods of making the above controlled-release compositions. Further disclosed are methods of treating overactive bladder or gastrointestinal disorders comprising administering a therapeutically effective amount of the controlled-release compositions described above.

These and other advantages of the compositions disclosed herein, as well as additional inventive features, will be apparent from the description of the invention provided herein.

### DETAILED DESCRIPTION

It has been surprisingly found that a suitable controlled-release tolterodine composition can be produced with a simpler coating and manufacture than the commercially available controlled-release compositions. In contrast to the commercially available controlled-release tolterodine formulations, it has been unexpectedly shown herein that a suitable controlled-release tolterodine dosage form can be produced from a core comprising two instead of three coatings. In one embodiment, a controlled-release tolterodine composition comprises a core comprising a water insoluble polymer, a first layer disposed on the core comprising tolterodine or a pharmaceutically acceptable salt thereof and a binder, and a second layer disposed on the first layer, wherein the second layer comprises a water insoluble polymer, a plasticizer, and a pore-forming agent.

The controlled-release tolterodine composition disclosed herein exhibits suitable dissolution profiles and/or reduced side effects. For example, the controlled-release tolterodine compositions can be administered once daily or even less frequently. The controlled-release tolterodine compositions provide optimal effective plasma levels of tolterodine over an extended period of time. In addition, the controlled-release tolterodine compositions maintain low, steady plasma peak values, for example, Cₘₐₓ, so as to reduce the incidence and severity of side effects such as dry mouth.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” is characterized by one or more pharmacokinetic parameters.

As used herein, “controlled-release” means a dosage form in which the release of the active agent is controlled or modified over a period of time. “Controlled” includes, for example, sustained, delayed, or pulsed release at a particular time. For example, controlled-release includes release of the active ingredient that is extended for longer than in an immediate release dosage form, i.e., at least over several hours. Controlled-release includes, for example, release over 12 or 24 hours.

As used herein, “dissolution profile” means a plot of the cumulative amount of active ingredient released as a function of time. The dissolution profile can be measured utilizing the Drug Release Test <724>, which incorporates standard test USP 26 (Test <711> Dissolution). A profile is characterized by the test conditions selected. Thus the dissolution profile can be generated at a pre-selected apparatus type, shaft speed, temperature, volume, and pH of the dissolution media.

A “dosage form” means a unit of administration of an active agent. Examples of dosage forms include tablets, capsules, injections, suspensions, liquids, emulsions, creams, ointments, suppositories, inhalable forms, transdermal forms, and the like.

As used herein, “oral dosage form” means a unit dosage form prescribed or intended for oral administration.

As used herein, “pharmaceutically acceptable” means that which is generally safe, non-toxic and neither biologically nor otherwise undesirable and includes that which are acceptable for veterinary use as well as human pharmaceutical use.

The inert core of the controlled-release composition comprises a water insoluble polymer. A water insoluble excipient is an excipient that has a water solubility of less than 1 part in 10,000 parts of water. In one embodiment, the core is substantially free of water soluble excipient, that is, that the core comprises less than 1 wt % of water soluble excipient. By using water insoluble polymer in the inert core, no sealcoat is necessary between the inert core and tolterodine-containing first layer to prevent potential chemical reactions between the core and active ingredient. The controlled-release composition disclosed herein is simplified compared to DETROL® LA, while maintaining a suitable controlled-release profile. This in turn has the benefits of a simplified manufacturing process and lower manufacturing cost. In one embodiment, the inert core consists essentially of a water insoluble polymer. By consists essentially of a water insoluble polymer, it is meant that the core comprises no excipients that substantially alter the water insolubility of the core.

Exemplary water insoluble polymers suitable as inert cores include, but are not limited to, microcrystalline cellulose (MCC), starch, ethyl cellulose, cellulose acetate, cellulose diacetate, cellulose triacetate, polyvinyl acetate, polymethacrylates, and a combination comprising at least one of the foregoing cores.

The choice of the inert core can have an effect on the release profile of the controlled-release composition. In one embodiment, the inert core comprises microcrystalline cellulose, for example, CELPHERE®. CELPHERE® is a 100% MCC spherical seed core. CELPHERE® is commercially available from Asahi Kasei Chemical Corporation in several different grades including CELPHERE® CP-708. CELPHERE® CP-708 has a mean particle size of 710 to 850 μm, a sphericity of 1.2, a bulk density of 0.93 g/cm³, a friability of 0%, and water absorption of 65%. CELPHERE® is high in mechanical strength and water absorption and is an optimum seed core for a wide range of aqueous layering and coating systems. In one embodiment, the inert core consists essentially of microcrystalline cellulose, for example, CELPHERE® CP-708.

In one embodiment, it may be desirable to reduce the particle size of the inert core to obtain a more uniform coated core and more consistent controlled-release performance. For example, the inert cores can be passed through a sieve to break agglomerates. In one embodiment, the inert cores are passed through a size 16 mesh to remove large particles. In another embodiment, the inert cores are passed through a size 35 mesh to obtain cores with desirable particle sizes.

Suitable particle sizes of the inert cores include, for example, about 50 μm to about 2,000 μm, or more specifically, about 100 μm to about 1,500 μm, or even more specifically, about 200 μm to about 1,200 μm, or still more specifically, about 500 μm to about 1,000 μm.

The inert core is present in an amount of about 50 to 95 weight percent (wt %), or more specifically, about 70 to 95 wt %, or even more specifically, about 75 to 90 wt %, based on the total weight of the controlled-release composition.

A first layer comprising tolterodine and a binder is disposed on the inert core. In one embodiment, the first layer is disposed directly on the core, that is, with no intervening layers between the inert core and the first layer. As used herein, the term “tolterodine” means tolterodine free base or its pharmaceutically acceptable salts, solvates (including hydrates), polymorphs, all optical isomers, or a combination comprising at least one of the foregoing forms of tolterodine. Tolterodine can be in crystalline or non-crystalline (amorphous) form.

As used herein, a “pharmaceutically acceptable salt” of tolterodine means a derivative of tolterodine, wherein tolterodine is modified by making non-toxic inorganic or organic acid addition salt thereof. Pharmaceutically acceptable inorganic salts include salts derived from inorganic acids such as hydrochloric, hydrobromic, sulfuric, sulfamic, phosphoric, nitric and the like. Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Combinations comprising at least one of the foregoing salts may also be used.

A particularly useful salt of tolterodine is tolterodine tartrate, or more specifically, tolterodine L-tartrate. Tolterodine tartrate can be prepared according to the process described in EP 325 571 or EP 667 852, EP 960 109, WO 01/49649, WO 03/14060, WO 98/03067, which are fully incorporated herein by reference.

Tolterodine is disposed onto the inert core in the presence of a binder. The binder adheres the tolterodine to the core particles, but preferably does not create such adhesive effects wherein particles of the selected composition adhere to one another. Suitable binders in the first layer include cellulose derivatives, such as, for example, hydroxypropyl cellulose, hydroxypropyl methyl cellulose (HPMC), carboxymethyl cellulose, methyl cellulose, hydroxyethyl cellulose, carboxyethyl cellulose, carboxymethylhydroxyethyl cellulose, and a combination comprising at least one of the foregoing cellulose derivatives. Suitable binders also include polyvinyl pyrrolidone (PVP), polyethylene glycol, polyvinyl alcohol, polymethacrylates, and a combination comprising at least one of the foregoing binders.

Exemplary hydroxypropyl cellulose polymers include, but are not limited to, polymers available under the trade names KLUCEL®, commercially available from Aqualon, and HPC, commercially available from Nippon Soda Co. Low viscosity KLUCEL® polymers include, but are not limited to, KLUCEL® EF, KLUCEL® LF, KLUCEL® JF, and KLUCEL® GF, wherein a 2 wt % aqueous solution has a viscosity of less than 1000 cPs. Medium viscosity KLUCEL® polymers include, but are not limited to, KLUCEL® ME, wherein a 2 wt % aqueous solution has a viscosity in the range from 4,000-6,500 cPs. Low viscosity HPC polymers include, but are not limited to, HPC-SL, HPC-L, and HPC-M, wherein 2 wt % aqueous solutions have viscosities of 3-6 cPs, 6-10 cPs, and 150-400 cPs, respectively. Medium viscosity HPC polymers include, but are not limited to, HPC-H, wherein a 2 wt % aqueous solution has a viscosity of 1,000-4000 cPs. In one embodiment, the binder comprises KLUCEL® LF.

The first layer optionally comprises a plasticizer to increase the flexibility of the binder. The added flexibility allows the binder and drug to adhere more efficiently and uniformly to the inert core. Suitable plasticizers include water-soluble or water-insoluble plasticizers. Exemplary water-soluble plasticizers include triethyl citrate, triacetin, polyethylene glycol, propylene glycol, sorbitol, glycerin, and combinations comprising at least one of the foregoing plasticizers. Exemplary water-insoluble plasticizers include dibutyl sebacate, diethyl phthalate, dibutyl phthalate, tributyl citrate, acetyl tributyl citrate, castor oil, mineral oil, glyceryl monostearate, and a combination comprising at least one of the foregoing plasticizers.

The first layer may optionally further comprise other excipients such as lubricating agents, for example, magnesium stearate, zinc stearate, talc, and colloidal silicon dioxide. When used, the lubricating agents are present in an amount of about 1 to 20 wt %, or more specifically, about 1 to 10 wt %, based on the total weight of the first layer.

In one embodiment, the first layer comprises tolterodine L-tartrate as active ingredient, hydroxypropyl cellulose, such as KLUCEL® LF, as binder, and optionally a plasticizer. In another embodiment, the first layer consists essentially of tolterodine L-tartrate as active ingredient and a binder. In yet another embodiment, the first layer consists essentially of tolterodine L-tartrate as active ingredient and hydroxypropyl cellulose, such as KLUCEL® LF, as binder.

The amounts of the tolterodine, the binder, and the optional plasticizer in the first layer are about 35 wt % to about 80 wt %, about 20 wt % to about 65 wt %, and 0 wt % to about 10 wt %, respectively, based on the total weight of the first layer.

It has been surprisingly found that the weight ratio of tolterodine to the binder in the first layer can have a significant effect on the release profile of the controlled-release composition. The weight ratio of tolterodine to the binder is about 5:1 to about 1:2, or more specifically, about 4:1 to about 1:1, or even more specifically, about 4:1 to about 2:1. A higher weight ratio of tolterodine to the binder is more desirable as more tolterodine can be loaded on to the inert core to increase bioavailability and obtain controlled-release of tolterodine. In one embodiment, the weight ratio of tolterodine to the binder is about 3:1.

The amount of the first layer is about 0.5 to 10 wt %, or more specifically, about 1 to 8 wt %, or even more specifically, about 1.5 to 6 wt %, based on the total weight of the controlled-release composition.

Following the application of the first layer on to the inert core, the resulting inert cores coated with the first layer are further coated with a second layer of a controlled-release coating. The second layer comprises a water insoluble polymer, a plasticizer, and a pore-forming agent.

It has been surprisingly found that by using a combination of a water insoluble polymer, a plasticizer, and a pore-forming agent in the second layer, desirable controlled-release profile of tolterodine is obtained. The selection of the water insoluble polymer, the pore-forming agent, the plasticizer, and the thickness of the second layer provide a particularly desirable dissolution profile of tolterodine at neutral and/or acidic pH-ranges.

Suitable water insoluble polymers for the second layer include, but are not limited to, ethyl cellulose, ethyl acrylate, methyl methacrylate, hydroxypropylmethyl cellulose phthalate, cellulose acetate phthalate, cellulose acetate trimellitate, or a combination comprising at least one of the foregoing polymers.

Suitable pore-forming agents include pH-independent and pH-dependent agents. Exemplary pH-independent pore-forming agents include polyvinyl pyrrolidone (also known as “PVP”, “povidone”, or “providone”), polyvinyl alcohol, hydroxyethyl cellulose, hydroxypropylmethyl cellulose, hydroxypropyl cellulose, methyl cellulose, carboxymethylcellulose, polyethylene glycol, and a combination comprising at least one of the foregoing pH-independent pore-forming agents.

In one embodiment, the pore-forming agent is a povidone, or more specifically, povidone K-30. Povidone polymers can be characterized by their viscosities in aqueous solutions, relative to that of water, expressed as K-values, of 10 to 120. The approximate weight average molecular weight for povidone K-30 is about 50,000.

Exemplary pH-dependent pore forming agents include acrylic acid or methacrylic acid/methacrylate based EUDRAGIT® polymers. Exemplary EUDRAGIT® polymers include, but not limited to, EUDRAGIT® L 100-55, EUDRAGIT® L 30D, EUDRAGIT® S, EUDRAGIT® FS 30D, and a combination comprising at least one of the foregoing pH-dependent pore-forming agents. EUDRAGIT® polymers are commercially available from Rohm Pharma GmbH, Germany.

Exemplary pH-dependent pore forming agents also include hydroxypropylmethyl cellulose phthalate, polyvinyl acetate phthalate, shellac, cellulose acetate trimellitate, cellulose acetate phthalate, and a combination comprising at least one of the foregoing pH-dependent pore-forming agents.

Suitable plasticizers for the second layer include, but are not limited to, diethyl phthalate, dibutyl phthalate, dibutyl sebacate, tributyl citrate, acetyl tributyl citrate, castor oil, mineral oil, glyceryl monostearate, and a combination comprising at least one of the foregoing plasticizers. It has been found that diethyl phthalate is particularly useful to provide desirable product performance, for example, suitable dissolution profile.

The second layer optionally further comprises other excipients such as lubricating agents, for example, magnesium stearate and talc, to improve process properties. When used, the lubricating agents are present in an amount of about 1 to 20 wt %, or more specifically, about 1 to 10 wt %, based on the total weight of the second layer.

In one embodiment, the second layer comprises a water insoluble polymer, a plasticizer, and a pore-forming agent. In another embodiment, the second layer consists essentially of a water insoluble polymer, a plasticizer, and a pore-forming agent. In yet another embodiment, the water insoluble polymer is ethyl cellulose, the plasticizer is diethyl phthalate, and the pore-forming agent is polyvinyl pyrrolidone.

The amounts of the water insoluble polymer, the plasticizer, and the pore-forming agent in the second layer are about 60 wt % to about 87 wt %, about 3 wt % to 10 wt %, and about 10 wt % to 30 wt %, respectively, based on the total weight of the second layer.

The amount of the second layer is about 5 to 20 wt %, or more specifically, about 7 to 15 wt %, or even more specifically, about 8 to 12 wt %, based on the total weight of the controlled-release composition. The thickness of the second layer is about 5 to 200 μm, or more specifically, about 10 to 100 μm, or even more specifically, about 10 to 50 μm.

The first layer and the second layer are applied onto the inert core and the inert core coated with the first layer, respectively, using techniques known in the art, for example, a fluidized bed or a coating pan.

Fluidized bed coaters, for example, bottom, tangential, or top-spray coaters, are suitable for spraying. A fluidized bed system, for example, from Wurster or Huettlin, is one in which an air jet is injected from underneath the bed, fluidizes the particles and effects a drying while the coating material is sprayed on to the core or coated core. To create pellets directly from powders and to further apply film coating on it, rotor or extrusion spheronisation systems may be employed.

A rotor granulation system (tangential spray fluidized bed) is a combination fluidized bed system fitted with a solid rotating disc at the base. In a rotor granulation process, centrifugal force, fluidization air velocity, and gravitational force all contribute to the efficiency of the coating process. In one typical rotor granulation process, tolterodine powder is deposited on an inert core in the presence of a binder solution. In another typical rotor granulation process, a suspension of tolterodine in a suspending agent is employed. The suspended or powder particles of tolterodine act to control the size of the atomized droplets. The suspended or powder tolterodine particles thus effectively act as a dusting powder to reduce agglomeration that can compete with tolterodine layering. In the absence of tolterodine particles, e.g., in the presence of tolterodine either wholly soluble or soluble to an appreciable extent in the binder solution, rotor-granulation is usually not performed due to stickiness, slow application rates, and/or formation of a rough surface texture on the particles.

In an exemplary process, a first layer coating mixture comprising tolterodine, a binder, an optional plasticizer or other excipients, and a solvent (solution) or dispersing agent (suspension) is prepared for coating the inert core. Suitable solvents and dispersing agents include water, ethanol, isopropyl alcohol, acetone, alcohol USP (95% ethanol, 5% water), denatured alcohol with methanol as a denaturant, for example, SDA-3A alcohol, and a combination comprising at least one of the foregoing solvents or dispersing agents.

In one embodiment, the solvent is a mixture of a denatured alcohol and water. It has been surprisingly found that the presence of small amount of water in the solvent advantageously allows for easier processing and spraying of tolterodine onto the core. The denatured alcohol and water in the solvent has a weight ratio of about 86:14 to about 94:6, or more specifically, about 88:12 to about 92:8.

In one embodiment, the first layer coating mixture is deposited onto the inert cores using a rotary granulation process. The first layer coating mixture comprising tolterodine and a binder, for example, KLUCEL® LF, is atomized onto a fluidized bed of inert cores located in the rotor granulator. Because of the difference in size between the cores and the atomized tolterodine and binder mixture, the tolterodine sticks to the cores and the binder retains the tolterodine on the cores. In the fluidized bed, a rotor-disk granulator makes the cores move with fluid-like motion. As the cores move within the fluidized bed, they are sprayed with the tolterodine and the binder mixture until the desired quantity of tolterodine is deposited onto the cores. Coating of tolterodine and the binder is optionally followed by a drying step.

The temperature, pressure, spraying rate, and plate speed are adjusted to minimize agglomeration of the cores and the tolterodine, and/or to allow for efficient tolterodine and core interaction. At high temperatures, the first layer coating mixture evaporates before adhering to the cores. At lower temperatures, however, the cores as well as the tolterodine can agglomerate, leading to the production of large agglomerates. The pressure in the spraying nozzle should be sufficient to atomize the first layer coating mixture, without making the atomized particles too small to adhere to the core substrate. Preferably, the pressure level retains the size of the atomized particles to act as their own anti-dusting agents, wherein agglomeration is limited. The spray rate can be optimized to prevent the first layer coating mixture from being lost during the processing, or excessive wetting.

The processing conditions are dependent upon many factors such as rotary granulator size and type. In a typical process, the inlet air temperature in the fluidized bed is about 35° C. to about 60° C., more specifically about 40° C. to about 50° C.; the dew point is about −5° C. to about 20° C., specifically about 0° C. to about 10° C.; the outlet air temperature is about 10° C. to about 40° C., specifically about 25° C. to about 32° C.; the product temperature is about 16° C. to about 40° C.; the atomizing air pressure is about 1 to 2 bar, specifically 1.5 bar; and the spraying rate is about 15 to 45 grams/minute, specifically about 20 to about 30 grams/minute. The plate speed is about 200 to about 400 rpm.

Once coated on the inert cores as the first layer, the content of the residual solvent or dispersing agent is negligible in the first layer, if not altogether absent, due to its removal during processing. For example, the content of the residual solvent or dispersing agent is less than 0.5 wt %, or more specifically, 0.1 wt %, based on the total weight of the core and the first layer.

The second layer of controlled-release coating is applied to the inert cores coated with the first layer using fluidized bed process as described above. Suitable solvents or dispersing agents are the same as those described above for preparing the first layer coating mixture.

In one embodiment, a second layer coating mixture is a solution and comprises a water insoluble polymer, a plasticizer, and a pore-forming agent in a solvent. In another embodiment, the solvent is alcohol, for example, ethanol. Typical spraying conditions are the same as for the first coating.

Optionally, the second layer coating is dried before applying a non-functional third layer coating, for example, a cosmetic coating. As used herein, “non-functional third layer coating” means that the coating is compatible with and does not substantially affect the dissolution properties of the controlled-release composition. The cosmetic coating optionally includes a colorant.

The controlled-release compositions described above are in the form of coated particles, granules, pellets, or spheres. These particles, pellets, granules, or spheres are ultimately processed in the form of tablets, capsules, or other dosage forms suitable for oral administration. In one embodiment, the controlled-release compositions, for example, coated particles, granules, pellets or spheres, are filled into hard gelatin capsule shells to provide a desired quantity of tolterodine in an oral dosage form.

Alternatively, the controlled-release compositions, for example, coated particles, granules, pellets or spheres, are made into tablets, for example, by first adding about 25 wt % to about 40 wt % of a pharmaceutically acceptable tablet excipient which will form a compressible mixture with the coated cores and which can be formed into a tablet without crushing the coated cores, and optionally an effective amount of a tablet disintegrating agent and a lubricant. Tablets are prepared by various techniques such as direct compression, wet granulation, or dry granulation. The tablets optionally are coated with a non-functional coating to form a nonfunctional layer.

The pharmaceutically acceptable tablet excipients include, but are not limited to, fillers, binders, diluents, disintegrants, anti-oxidants, lubricants/glidants, and a combination comprising at least one of the foregoing excipients. Suitable fillers include, but are not limited to, lactose, dextrose, mannitol, calcium phosphate, microcrystalline cellulose, kaolin, powdered sucrose, and combinations comprising at least one of the foregoing excipients. Suitable disintegrants include, but are not limited to, crospovidone, croscarmellose sodium, dry starch, sodium starch glycolate, and the like, and a combination comprising at least one of the foregoing disintegrants. Suitable lubricants include, but are not limited to, calcium stearate, glycerol behenate, magnesium stearate, mineral oil, polyethylene glycol, sodium stearyl fumarate, stearic acid, talc, vegetable oil, zinc stearate, and a combination comprising at least one of the foregoing lubricants.

The controlled-release tolterodine compositions described above exhibit desirable dissolution profiles. In one embodiment, the compositions exhibit a dissolution profile such that after 1 hour, about 1 to about 30%, or more specifically, about 1 to about 20%, of the tolterodine is released; after 3 hours, about 40 to about 85%, or more specifically, about 40 to about 70% of the tolterodine is released; and after 7 hours, about 50 to about 100%, or more specifically, about 50 to about 95% of the tolterodine is released, wherein the dissolution is performed with 900 ml of pH 6.8 phosphate buffer at 37° C. in USP Type 1 Apparatus (USP, <711> Dissolution), at a paddle speed of 100 rpm.

In another embodiment, the compositions exhibit a dissolution profile such that after 1 hour, about 1 to about 30%, or more specifically, about 1 to about 20%, of the tolterodine is released; after 3 hours, about 20 to about 50%, or more specifically, about 20 to about 40% of the tolterodine is released; and after 7 hours, about 35 to about 70%, or more specifically, about 40 to about 70% of the tolterodine is released, wherein the dissolution is performed with 900 ml of 0.1N HCl at 37° C. in USP Type 1 Apparatus (USP, <711> Dissolution), at a paddle speed of 100 rpm.

In another embodiment, the controlled-release compositions described above exhibit a dissolution profile that is substantially the same as that of DETROL® LA. As used herein, “DETROL® LA” means tolterodine tartrate capsule compositions manufactured by Pfizer, Inc., a drug product associated with New Drug Application Number 021228. Specifically, DETROL® LA means hard gelatin extended release capsules of tolterodine tartrate, equivalent of 2 and 4 mg of tolterodine L-tartrate, in the presence of inactive ingredients of sucrose, starch, hypromellose, ethylcellulose, medium chain triglycerides, oleic acid, gelatin, and FD&C Blue #2. The 2-mg capsules also contain yellow iron oxide. Both capsule strengths are imprinted with a pharmaceutical grade printing ink that contains shellac glaze, titanium dioxide, propylene glycol, and simethicone. As used herein, “substantially the same” means that the dissolution data for the controlled-release compositions described above at 1, 3, and 7 hours are within 80-125% that of DETROL® LA when measured under the same dissolution test conditions.

Also disclosed herein are methods of preparing controlled-release compositions of tolterodine. In one embodiment, the method comprises a) providing an inert core of a water insoluble polymer; b) applying a first layer comprising tolterodine or a pharmaceutically acceptable salt thereof and a binder onto the inert core; and c) applying a second layer comprising a water insoluble polymer, a plasticizer, and optionally a pore-forming agent onto the first layer.

Further disclosed herein are methods of treating overactive bladder and gastrointestinal disorders, wherein the method comprises administering a therapeutically effective amount of the controlled-release compositions described above.

The following examples further illustrate the invention but should not be construed as in any way limiting its scope. In particular, the processing conditions are merely exemplary and can be readily varied by one of ordinary skill in the art.

### EXAMPLES

### Example 1

### Controlled-Release Tolterodine Tartrate Particles—Small Batch

Water insoluble CELPHERE® CP-708 spheres (700 g/batch) were used as the inert cores of the controlled-release tolterodine L-tartrate composition. The CELPHERE® CP-708 spheres were passed through a size 16 mesh to remove large particles. Next, screened spheres were further passed through a size 35 mesh to obtain inert cores with mean particle sizes of about 700 to about 850 μm for coating with the first layer.

A first layer coating mixture was prepared by dissolving appropriate amounts of tolterodine L-tartrate and KLUCEL® LF in a solvent mixture comprising denatured alcohol and water. The composition of the first layer coating mixture is shown in Table 1.

TABLE 1
First Layer Coating Mixture Composition

| Ingredient | Amount/Batch, g |
|---|---|
| Tolterodine Tartrate | 21 |
| KLUCEL ® LF | 7 |
| Denatured Alcohol, | 602 |
| USP¹ | |
| Water¹ | 98 |
| Total | 728 |

¹This ingredient is substantially not present in the end product of inert cores coated with the first layer.

The inert cores coated with the first layer (tolterodine tartrate and KLUCEL®) were prepared in the following manner. First, a first layer coating mixture was prepared by dissolving KLUCEL® LF (7 g) and tolterodine (21 g) in denatured alcohol (602 g) and water (98 g). Then, screened CELPHERE® CP-708 (microcrystalline cellulose) particles (700 g) having mean particle sizes of about 700 to 850 μm were loaded in the product container of 4″ Wurster rotor granulator. The first layer coating mixture was then sprayed onto the CELPHERE® CP-708 particles. The coating conditions are shown in Table 2. After the completion of coating, the resulting coated cores were dried at 50° C. in the Wurster for 10 minutes.

TABLE 2
Spraying Conditions (4″ Wurster) for First Layer Coating

| Condition | Condition | |
|---|---|---|
| Product Temperature | 26-32° | C. |
| Process Air Volume | 45-50 | cfm |
| Inlet Temperature | 48-50° | C. |
| Inlet Air Dew Point | 5-10° | C. |
| Outlet Temperature | 25-32° | C. |
| Atom Air Press | 1.5 | bar |
| Spray Rate | 20-25 | g/min |
| Total Air Volume | 120-140 | cfm |

The inert cores coated with the first layer were then coated with a second layer of a controlled-release coating. The composition of the second layer coating mixture is shown in Table 3.

TABLE 3
Second Layer Coating Mixture Composition For Example 1

| Ingredient | Amount/Batch, g | Weight %, composition |
|---|---|---|
| Ethyl Cellulose N45 | 104 | 70.4 |
| Polyvinyl pyrrolidone | 36.5 | 24.7 |
| (PVP) K30 | | |
| Diethyl phthalate | 7.2 | 4.9 |
| Denatured Alcohol, | 1,905 | |
| USP¹ | 0 | |
| Total | 2,052.7 | |

¹This ingredient is not present in the end product of the coated controlled-release compositions.

Controlled-release tolterodine tartrate particles were prepared in the following manner. A second layer coating mixture was prepared by dissolving ethyl cellulose N45 (104 g), PVP K30 (36.5 g), and diethyl phthalate (7.2 g) in alcohol (1,905 g). The inert cores coated with the first layer obtained in Example 1 (700 g) were loaded in the product container of GPCG-3 granulator fitted with 4″ Wurster. Next, the second layer coating mixture was sprayed onto the inert cores coated with the first layer. The spraying conditions used are summarized in Table 2. The coated samples were dried at 50° C. in the Wurster for 10 minutes.

The spray coating of the second layer was stopped at different times to obtain different amounts of the second layer. For example, controlled-release particles having about 9 wt %, 13 wt %, and 15 wt %, respectively, of the second coating based on the total weight of the inert cores coated with the first layer, were obtained for dissolution release testing.

Dissolution tests were performed using the controlled-release particles obtained above using USP Type 1 apparatus, phosphate buffer (pH 6.8, 900 mL) as the dissolution medium, and the rotation speed at 100 rpm. The temperature during the dissolution test was 37±0.5° C. As a comparison, DETROL® LA was also tested. The dissolution results using pH 6.8 phosphate buffer as the dissolution medium are summarized in Table 4.

TABLE 4
Dissolution data of controlled-release
particles using pH 6.8 phosphate buffer

| | Cumulative release of tolterodine tartrate % of theoretical | | | |
|---|---|---|---|---|
| | amount of tolterodine tartrate in composition, wt % | | | |
| | 9 wt % | 13 wt % | 15 wt % | |
| Time, | second | second | second | DETROL ® |
| Hour | layer coating | layer coating | layer coating | LA |
| 0.5 | 4 | 0.4 | 0.2 | 6 |
| 1 | 15.7 | 2.5 | 1 | 14.3 |
| 2 | 39.9 | 11.4 | 6.2 | 40 |
| 3 | 58.7 | 22 | 13.3 | 62 |
| 5 | 79.1 | 42.6 | 29.2 | 84.3 |
| 7 | 87.8 | 57.8 | 43.7 | 90.3 |
| 9 | 91.8 | 67.7 | 54.7 | 92.7 |
| 15 | 96.7 | 79.2 | 71.1 | 95 |
| 20 | 98.5 | 83 | 76 | 96 |

Dissolution tests using 0.1N HCl as the dissolution medium were also conducted and the results are summarized in Table 5.

TABLE 5
Dissolution data of controlled-release
particles (small batch) using 0.1N HCl

| | Cumulative release of tolterodine tartrate % of theoretical | | | |
|---|---|---|---|---|
| | amount of tolterodine tartrate in composition, wt % | | | |
| | 9 wt % | 13 wt % | 15 wt % | |
| Time, | second | second | second | DETROL ® |
| Hour | layer coating | layer coating | layer coating | LA |
| 0.5 | 8.3 | 2.4 | 0.9 | 11 |
| 1 | 15.3 | 7.9 | 4.7 | 16.3 |
| 2 | 26.4 | 15.4 | 11.3 | 24.3 |
| 3 | 35.4 | 21 | 15.6 | 32.7 |
| 5 | 52.4 | 30.6 | 22.8 | 49.3 |
| 7 | 66.2 | 39.4 | 29 | 62.7 |
| 9 | 77.2 | 47.3 | 34.8 | 75.3 |
| 15 | 98 | 68.1 | 50.7 | 94.7 |
| 20 | 105 | 80.8 | 61.9 | 100.3 |

It can be seen from Tables 4 and 5 that controlled-release compositions having different amounts of the second coating had different in vitro release dissolution profiles in pH 6.8 phosphate medium and 0.1N HCl medium. The cumulative release of tolterodine tartrate generally decreases as the amount of the second layer release rate controlling coating increases from 9 wt % to 15 wt %, based on the weight of the inert cores and the first layer. The controlled-release composition having about 9 wt % of the second layer, based on the weight of the inert cores and the first layer, had substantially the same dissolution profile as that of DETROL® LA.

### Example 2

### Controlled-Release Tolterodine Tartrate Particles—Large Batch

In this example, a larger batch was used to prepare controlled-release tolterodine tartrate particles. Water insoluble CELPHERE® CP-708 spheres (15,000 g/batch) were used as the inert cores of the controlled-release tolterodine composition. The first layer was applied to the CELPHERE® CP-708 spheres cores using the same process as in Example 1. The composition of the first layer coating mixture is shown in Table 6.

TABLE 6
First Layer Coating Mixture Composition for Example 2

| Ingredient | Amount/Batch, g |
|---|---|
| Tolterodine Tartrate | 450 |
| KLUCEL ® LF | 150 |
| Denatured Alcohol, | 12,900 |
| USP¹ | |
| Water¹ | 2,100 |
| Total | 15,600 |

¹This ingredient is substantially not present in the end product of inert cores coated with the first layer.

The inert cores coated with the first layer (tolterodine tartrate and KLUCEL®) were prepared in the following manner. First, a first layer coating mixture was prepared by dissolving KLUCEL® LF and tolterodine L-tartrate in denatured alcohol and water. Then, screened CELPHERE® CP-708 (microcrystalline cellulose) particles having mean particle sizes of about 700 to 850 μm were loaded in the product container of GPCG-5 rotor granulator (50 cm rotor). The first layer coating mixture was then sprayed onto the CELPHERE® CP-708 particles. The coating conditions are shown in Table 2. After the completion of coating, the resulting coated cores were dried at 50° C. for 15 minutes.

The inert cores coated with the first layer obtained above were coated with a second layer of controlled-release coating. The composition of the second layer coating mixture is shown in Table 7.

TABLE 7
Second Layer Coating Mixture Composition For Example 2

| Ingredient | Amount/Batch, g | Weight % of Second Layer |
|---|---|---|
| Ethyl Cellulose N45 | 499 | 70.3 |
| Polyvinyl pyrrolidone | 175 | 24.7 |
| (PVP) K30 | | |
| Diethyl phthalate | 36 | 5.0 |
| Denatured Alcohol, | 0 | 0 |
| USP¹ | 0 | 0 |
| Total | 710 | 100 |

¹This ingredient is not present in the end product of the coated controlled-release compositions.

Controlled-release tolterodine tartrate particles were prepared in the following manner. A second layer coating mixture was prepared by dissolving ethyl cellulose N45, PVP K30, and diethyl phthalate in alcohol. The inert cores coated with the first layer obtained above were loaded in the product container of a 7″ Wursterr. Next, the second layer coating mixture was sprayed onto the inert cores coated with the first layer. The spraying conditions used are summarized in Table 2. The coated samples were dried at 50° C. for 15 minutes.

The spray coating of the second layer was stopped at different times to obtain different amount of the second layer. For example, controlled-release particles having about 9 wt %, 11 wt %, and 13 wt %, respectively, of the second coating based on the total weight of the inert cores coated with the first layer, were obtained for dissolution release testing.

Dissolution tests were performed using the controlled-release particles obtained above using USP Type 1 apparatus, phosphate buffer (pH 6.8, 900 mL) as the dissolution medium, and the rotation speed at 100 rpm. The temperature during the dissolution test was 37±0.5° C. As a comparison, DETROL® LA was also tested. The dissolution results using pH 6.8 phosphate buffer as the dissolution medium are summarized in Table 8.

TABLE 8
Dissolution data of controlled-release particles
(large batch) using pH 6.8 phosphate buffer

| | Cumulative release of tolterodine tartrate % of theoretical | | | |
|---|---|---|---|---|
| | amount of tolterodine tartrate in composition, wt % | | | |
| | 9 wt % | 11 wt % | 13 wt % | |
| Time, | second | second | second | DETROL ® |
| Hour | layer coating | layer coating | layer coating | LA |
| 0.5 | 4 | 1.3 | 2 | 6 |
| 1 | 15.7 | 8.8 | 9 | 14.3 |
| 2 | 39.9 | 29.1 | 38 | 40 |
| 3 | 58.7 | 46.8 | 62 | 62 |
| 5 | 79.1 | 69.7 | 80 | 84.3 |
| 7 | 87.8 | 78.8 | 88 | 90.3 |
| 9 | 91.8 | 83.3 | 92 | 92.7 |
| 15 | 96.7 | 90.5 | 98 | 95 |
| 20 | 98.5 | 91.5 | 101 | 96 |

The dissolution tests using 0.1N HCl as the dissolution medium were also conducted and the results are summarized in Table 9.

TABLE 9
Dissolution data of controlled-release particles using 0.1N HCl

| | Cumulative release of tolterodine tartrate % of theoretical | | | |
|---|---|---|---|---|
| | amount of tolterodine tartrate in composition, wt % | | | |
| | 9 wt % | 11 wt % | 13 wt % | |
| Time, | second | second | second | DETROL ® |
| Hour | layer coating | layer coating | layer coating | LA |
| 0.5 | 8.3 | 5.4 | 7 | 11 |
| 1 | 15.3 | 13 | 16 | 16.3 |
| 2 | 26.4 | 22.9 | 28 | 24.3 |
| 3 | 35.4 | 31.6 | 39 | 32.7 |
| 5 | 52.4 | 47.6 | 59 | 49.3 |
| 7 | 66.2 | 60.5 | 76 | 62.7 |
| 9 | 77.2 | 71.4 | 90 | 75.3 |
| 15 | 98 | 91.6 | 104 | 94.7 |
| 20 | 105 | 99 | 106 | 100.3 |

It can be seen from Tables 8 and 9 that controlled-release compositions having different amounts of the second coating had different in vitro release dissolution profiles in Ph 6.8 phosphate medium and 01N HCl medium. The controlled-release composition having about 9 wt % of the second layer, based on the weight of the inert cores and the first layer, had substantially the same dissolution profile as that of DETROL® LA.

### Example 3

### Hard Gelatin Capsules

The controlled-release tolterodine L-tartrate particles obtained in Examples 1 and 2 are filled into hard gelatin capsules to give 2 and 4 mg tolterodine L-tartrate salt equivalent doses, respectively.

### Example 4

### Tablets

The controlled-release tolterodine particles obtained in Examples 1 and 2 are mixed with a compressible excipient and the resulting mixture is compressed into tablets to give 2 and 4 mg tolterodine L-tartrate salt equivalent doses, respectively.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A controlled-release composition, comprising:
(i) an inert core comprising a water insoluble polymer;
(ii) a first layer disposed on the inert core, wherein the first layer comprises tolterodine or a pharmaceutically acceptable salt thereof and a binder; and
(iii) a second layer disposed on the first layer, wherein the second layer comprises a water insoluble polymer, a plasticizer, and a pore-forming agent.

2. The controlled-release composition of claim 1, wherein the first layer is disposed directly onto the inert core.

3. The controlled-release composition of claim 1, wherein the controlled-release composition exhibits a dissolution profile such that after 1 hour, about 1 to about 30% of the tolterodine is released; after 3 hours, about 40 to about 85% of the tolterodine is released; and after 7 hours, about 50 to about 100% of the tolterodine is released, wherein the dissolution is performed with 900 ml of pH 6.8 phosphate buffer at 37° C. in USP Type 1 Apparatus (USP, <711> Dissolution), at a paddle speed of 100 rpm.

4. The controlled-release composition of claim 1, wherein the controlled-release composition exhibits a dissolution profile that is substantially the same as that of DETROL® LA.

5. The controlled-release composition of claim 1, wherein the water insoluble polymer in the inert core is microcrystalline cellulose.

6. The controlled-release composition of claim 1, wherein the binder in the first layer is hydroxypropyl cellulose, hydroxypropylmethyl cellulose, carboxymethyl cellulose, methyl cellulose, hydroxyethyl cellulose, carboxyethyl cellulose, carboxymethylhydroxyethyl cellulose, polyvinyl pyrrolidone, polyethylene glycol, polyvinyl alcohol, polymethacrylates, or a combination comprising at least one of the foregoing polymers.

7. The controlled-release composition of claim 1, wherein the binder in the first layer is hydroxypropyl cellulose.

8. The controlled-release composition of claim 1, wherein the weight ratio between tolterodine and the binder in the first layer is about 5:1 to about 2:1.

9. The controlled-release composition of claim 1, wherein the weight ratio between tolterodine and the binder in the first layer is about 4:1 to 2:1.

10. The controlled-release composition of claim 1, wherein the water insoluble polymer in the second layer is ethyl cellulose, ethyl acrylate, methyl methacrylate, hydroxypropylmethyl cellulose phthalate, cellulose acetate phthalate, cellulose acetate trimellitate, or a combination comprising at least one of the foregoing polymers.

11. The controlled-release composition of claim 10, wherein the water insoluble polymer in the second layer is ethyl cellulose.

12. The controlled-release composition of claim 1, wherein the plasticizer in the in the second layer is diethyl phthalate, dibutyl phthalate, dibutyl sebacate, tributyl citrate, acetyl tributyl citrate, castor oil, mineral oil, glyceryl monostearate, and a combination comprising at least one of the foregoing plasticizers.

13. The controlled-release composition of claim 12, wherein the plasticizer is diethyl phthalate.

14. The controlled-release composition of claim 1, wherein the pore-forming agent is polyvinyl pyrrolidone, polyvinyl alcohol, hydroxyethyl cellulose, hydroxypropylmethyl cellulose, hydroxypropyl cellulose, methyl cellulose, carboxymethylcellulose, polyethylene glycol, or a combination comprising at least one of the foregoing polymers.

15. The controlled-release composition of claim 14, wherein the pore-forming agent is polyvinyl pyrrolidone.

16. The controlled-release composition of claim 1, wherein the second layer comprises a combination of ethyl cellulose, diethyl phthalate, and polyvinyl pyrrolidone.

17. The controlled-release composition of claim 1, wherein the amounts of the water insoluble polymer, the plasticizer, and the pore-forming agent in the second layer are about 65 wt % to about 75 wt %, about 3 wt % to 10 wt %, and about 10 wt % to 30 wt %, respectively, based on the total weight of the second layer coating.

18. The controlled-release composition of claim 1, wherein the amount of the second layer is about 5 wt % to about 20 wt % based on the total weight of the controlled-release composition.

19. The controlled-release composition of claim 1, wherein the amount of the second layer is about 9 wt % to 11 wt %, based on the total weight of the controlled-release composition.

20. A controlled-release composition, comprising:
(i) an inert core consisting essentially of a water insoluble polymer;
(ii) a first layer coated on the inert core, wherein the first layer consists essentially of tolterodine or a pharmaceutically acceptable salt thereof and a binder; and
(iii) a second layer coated on the first layer, wherein the second layer consists essentially of a water insoluble polymer, a plasticizer, and a pore-forming agent.

21. A method of producing a controlled-release composition of claim 1, wherein the method comprises:
a) providing an inert core of a water insoluble polymer;
b) applying a first layer comprising tolterodine or a pharmaceutically acceptable salt thereof and a binder onto the inert core; and
c) applying a second layer comprising a water insoluble polymer, diethyl phthalate, and optionally a water soluble polymer onto the first layer;
wherein the tolterodine or a pharmaceutically acceptable salt thereof and the binder in step (b) are dissolved in a solvent comprising an alcohol and water for application.

22. The method of claim 21, wherein the alcohol and the water are present in a weight ratio of about 86:14 to about 94:6.
